(19) United States
(12) Patent Application Publication
(10) Pub. No.: US 2023/0135738 A1
(43) Pub. Date: May 4, 2023

(54) TISSUE ANALYSIS DEVICE AND TISSUE ANALYSIS METHOD FOR CHARACTERIZING PROSTATE CANCER WITH MICROWAVES

(71) Applicant: ISTANBUL TEKNIK UNIVERSITESI, Istanbul (TR)

(72) Inventors: Ibrahim AKDUMAN, Istanbul (TR); Mehmet CAYOREN, Istanbul (TR); Tuba YILMAZ ABDOLSAHEB, Istanbul (TR)

(73) Assignee: ISTANBUL TEKNIK UNIVERSITESI, Istanbul (TR)

(21) Appl. No.: 17/802,563

(22) PCT Filed: Jan. 12, 2021
(86) PCT No.: PCT/TR2021/050017

§ 371 Date: Aug. 26, 2022

(30) Foreign Application Priority Data
Feb. 28, 2020 (TR) 2020/03070

(51) Int. Cl. A61B 5/0507; A61B 5/00

(52) U.S. Cl. CPC A61B 5/0507; A61B 5/4381

(57) ABSTRACT

A device has been developed for diagnosing malignant prostate tissue to be utilized in the healthcare sector as a diagnostics equipment. The tissue identification device of the present invention includes a probe or resonator, a measurement tool including narrow band antennas, a classification unit, an inverse problem calculation unit, an S parameter measurement unit, and calculation unit (computer). The present device enables accurate diagnosis of prostate cancer by means of the above-mentioned elements it includes.

### CROSS REFERENCE TO THE RELATED APPLICATIONS

This application is the national phase entry of International Application No. PCT/TR2021/050017, filed on Jan. 12, 2021, which is based upon and claims priority to Tukish Patent Application No. 2020/03070, filed on Feb. 28, 2020, the entire contents of which are incorporated herein by reference.

### TECHNICAL FIELD

The invention is related to a device and a tissue analysis method that has been developed for diagnosing malignant prostate tissue to be used in the health sector and for enabling accurate diagnosis of prostate cancer.

### BACKGROUND

Currently, the malignant tumours in prostate tissues are diagnosed with a standard protocol which includes a hand examination by a physician as the first step. Next, if a tumour is diagnosed during the hand examination, the patient receives a biopsy that entails removing part of the tissue or tissue fluid from the tumour site. Lastly, the removed sample is sent for pathological analysis to determine whether the suspected tumour tissue is malignant or benign. Biopsy and pathology requires specialized equipment and trained staff to perform the necessary tasks. Access to such facilities and availability of specialized staff can be limited in rural areas. Additionally, based on the demand for pathological analysis, it can take from few days to few weeks to receive a diagnosis report from the centers/clinics. To this end, the current diagnosis protocol/process is costly and time-consuming. Also, the protocol is entirely human controlled, thus, the diagnosis is vulnerable to human error. Therefore, alternative technologies that can encounter the current challenges of the prostate cancer diagnostics process are needed. Microwave diagnosis and treatment devices have recently been proposed as alternative technologies that can be used for imaging or treatment of many different tumour tissues. The working principle of these technologies depends on the inherent dielectric property (relative permittivity and conductivity) discrepancies between the malign and benign tissues at microwave frequencies.

Dielectric properties of a material can be function of different variables including temperature, frequency, and material structure. These properties are either used as design parameters or they are retrieved to image buried objects or diagnose a tumour. The dielectric properties of tissues at microwave frequencies are traditionally measured with commercial open-ended coaxial probes in a laboratory environment. These probes are widely preferred for characterization of biological tissue dielectric properties due to broadband measurement capabilities and limited sample preparation requirements. Due to these characteristics, open-ended coaxial probes can be used for classifying biological materials belonging to different tissue categories. However, the commercial open-ended coaxial probes are error prone and suffer from low measurement repeatability. Hence, the open-ended coaxial probes cannot be deployed to clinics and operation rooms for practical use. To enable utilization of these probes in practical applications, such as diagnosis of prostate cancer, there is a need to increase the measurement accuracy and repeatability rates.

Current state of the art in prostate cancer diagnosis is summarized as follows,

In the United States Patent document numbered U.S. Pat. No. 5,829,437A of the prior art, a system and a method for determining the tumour type, growing in normal human tissues, was proposed. The method uses the relative dielectric property discrepancy between the tissues for tumour characterization.

In the European patent document numbered EP2465428A1 of the prior art, electromagnetic based detection systems for determining anomalies (for example tumours and calcifications) in tissues was presented. Particularly, the proposed device targets the early diagnosis of breast, colorectal, and prostate cancer.

In the United States patent document numbered U.S. Pat. No. 5,369,251A of the prior art, the development and utilization of needle type microwave interstitial probe antennas were disclosed for the selective storage of microwave power in dielectric mediums, especially in biological tissues for both in vivo and in vitro applications.

In the United States patent document numbered US2011152853A1 of the prior art, an electromagnetic based surgical ablation system and the related operation method was disclosed. The system includes a generator that can provide selective electromagnetic power to an ablation probe.

In the United States patent document numbered US2013144554A1 of the prior art, a system configuration used for determining and evaluating the status of materials was given. The proposed system employs signal processing techniques and works in a wide number of frequencies. Also, the technique is claimed to work with relatively less loss in comparison to the previously proposed similar technologies.

In the United States patent document numbered US2016073923A1 of the prior art, design of a microwave resonance circuit printed on a dielectric substrate and designed to be used as a feeding circuit for a probe was disclosed. The probe was aimed to be used for collecting dielectric property measurement data from the animal and human tissues.

In the United States Patent document numbered US2007179397A1 of the prior art, probes, systems, and methods for tissue characterization with dielectric properties was disclosed.

Based on the analysis of devices developed for the diagnosis of malignant prostate tissues of the prior art, we can state that a new technique is needed that can potentially either be used to aid pathology or to replace pathology by integrating an automated decision making mechanism during biopsy application. Microwave based tissue characterization technique is a candidate alternative technology for pathology that can potentially eliminate the need for human assistance and enable rapid, real time, diagnostics. However, it can only be realized through reducing the inherent measurement errors via adoption of classification algorithms that can potentially broaden the usable frequency range for commercially available open-ended coaxial probes, decrease the method-specific error rates, and eliminate the calibration degradation based errors as well as errors due to SMA connection problems. Moreover, the devices of the prior art lead to high error rates and therefore novel embodiments are required that shall eliminate the disadvantages mentioned above. Such approach can provide a solution to present system drawbacks.

### SUMMARY

The aim of the invention is to provide a new device and operation method thereof that functions with a novel automated decision-making mechanism that eliminates the disadvantages of the prior systems, that enables to reduce the error rates of the measurement by using dielectric property data in the surgical environment and/or S parameter measurements, and by the adaptation of classification algorithms that can characterize the malign and/or benign and healthy prostate tissues with inherent dielectric property discrepancies.

The aim of the invention is to develop a new device that shall enable to reduce error rates following measurement, by the adaptation of classification algorithms and a method of operation of said device.

Another aim of the invention is to provide a new device and operation method thereof, where the measurement errors arising from cable movements are minimized.

Another aim of the invention is to develop a new device that enables to reduce error rates of the measurement, by the adoption of classification algorithms and a method of operation of said device.

Another aim of the invention is to provide a new device and operation method thereof, that can calculate dielectric characteristics with high precision from S parameters, using mathematical approaches based on inverse problem solutions.

The proposed device for the diagnosis of malignant prostate tissues that is provided to meet the aims of this invention has been illustrated in the attached figures.

### BRIEF DESCRIPTION OF THE DRAWINGS

FIG. 1: The flow chart illustrating the measurement principle of the proposed tissue identification device subject to the invention.

FIG. 2: General perspective view of the device subject to the invention.

FIG. 3: General view of the open-ended coaxial probe produced with a permanent connection with the RF/microwave cable (SMA connection is removed) located in the device subject to the invention.

FIG. 4: Illustration of the single port contact probe aperture opening within the device subject to the invention.

FIG. 5: Cross section view of the dual port contact probe aperture opening within the device subject to the invention.

FIG. 6: View of the open-ended contact probe located in the device and biopsy guide subject to the invention.

FIG. 7: View of the planar micro-strip resonators that can be integrated, instead of open-ended coaxial probes, to the device subject to the invention.

FIG. 8: View of the microstrip resonators that can be integrated, instead of open-ended coaxial probes, to the device subject to the invention.

FIG. 9: Top view of the narrow band planar microstrip resonators can be integrated, instead of open-ended coaxial probes, to the device subject to the invention.

FIG. 10: Side view of the narrow band planar microstrip resonators that can be integrated, instead of open-ended coaxial probes, to the device subject to the invention.

FIG. 11: Top view of the wide band planar microstrip resonators that can be integrated, instead of open-ended coaxial probes, to the device subject to the invention.

FIG. 12: The view of the prob directly connected to S parameter measurement unit, without the RF cable, located in the device subject to the invention.

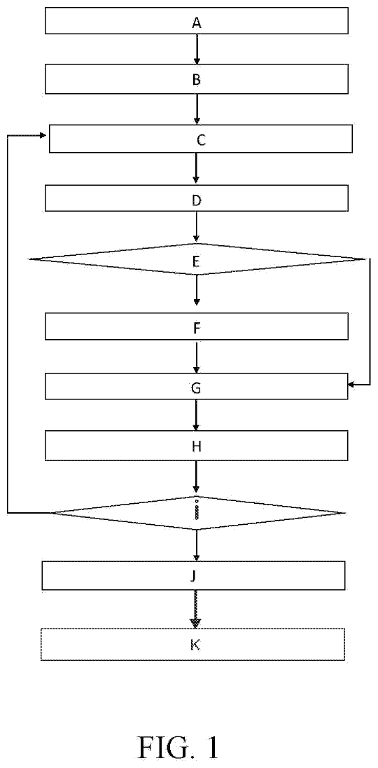

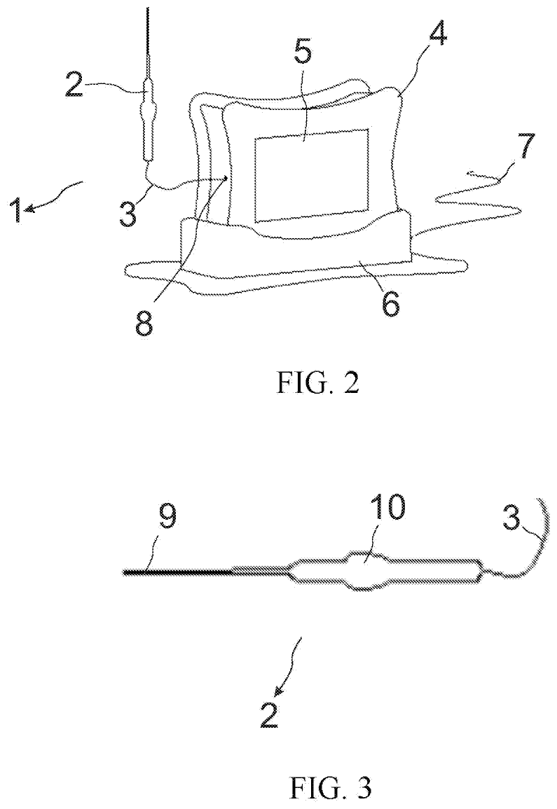

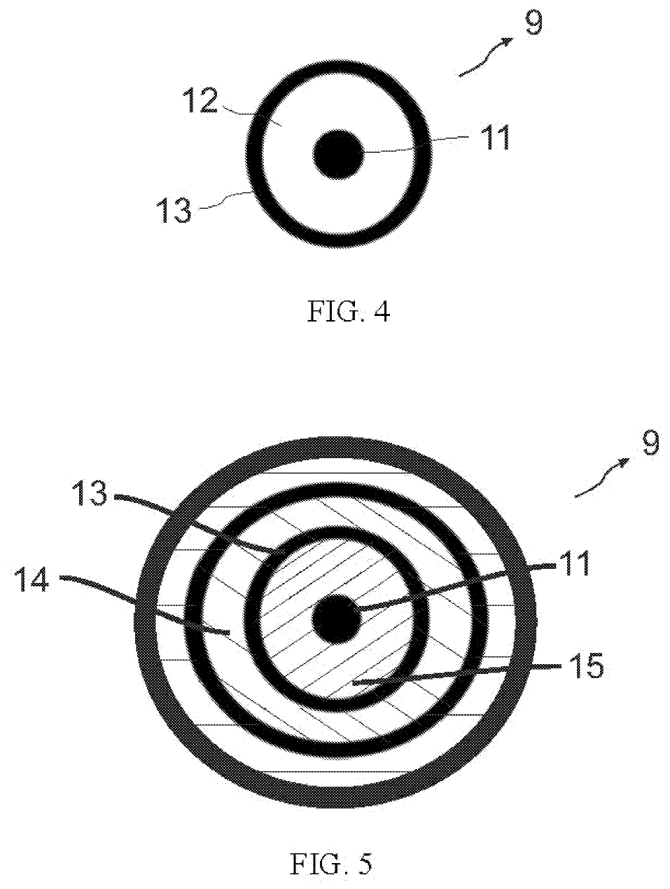

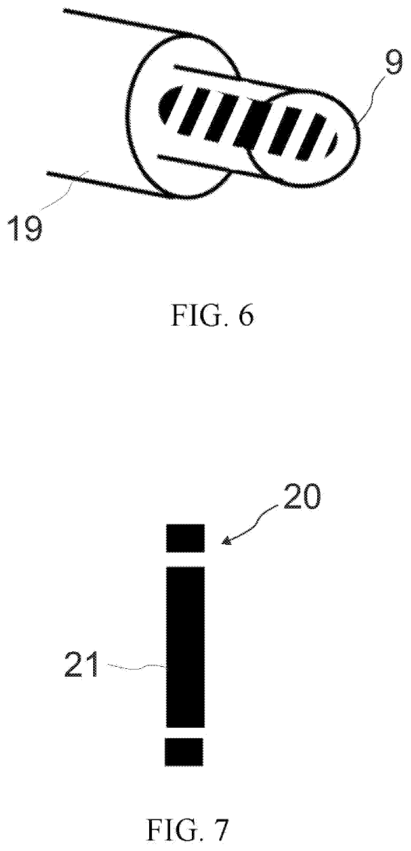

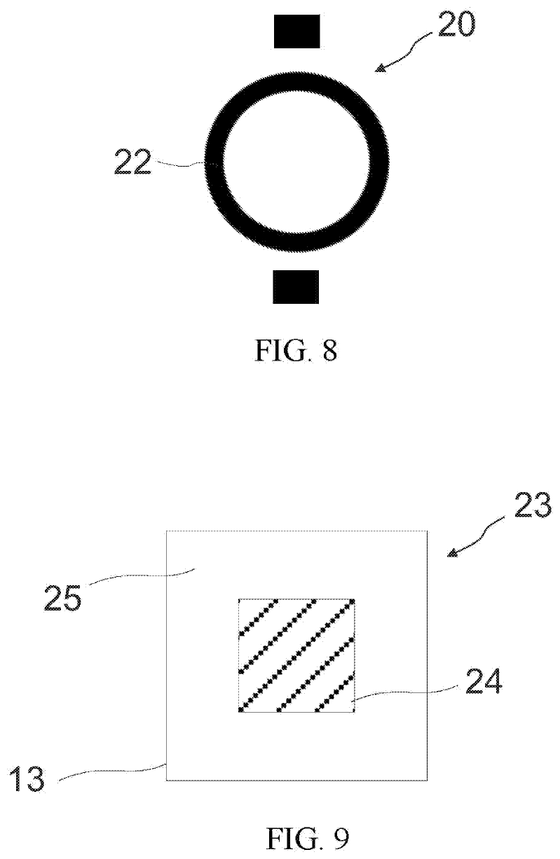

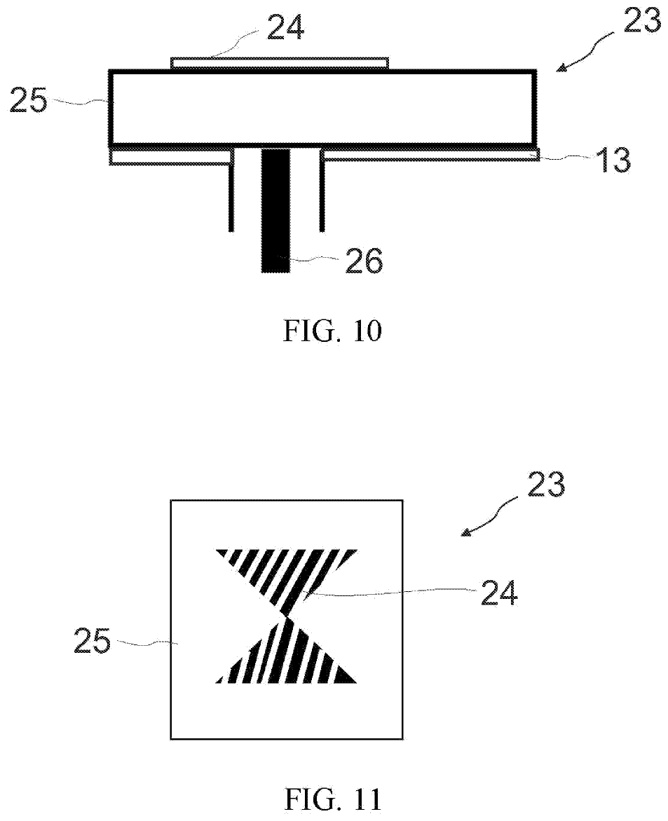

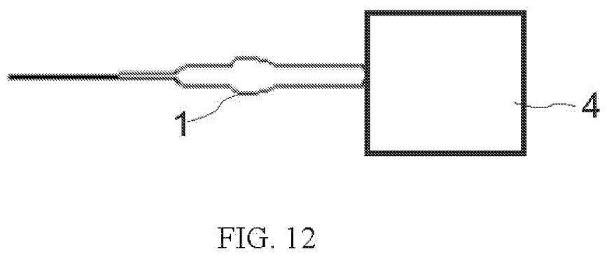

The parts in the figures have each been numbered and their references have been listed below.

1. Tissue Identification/characterization device

2. Measurement tool

3. RF/Microwave Cable

4. S Parameter Measurement Unit and Calculation/Retrieval Unit (computer)

5. Digital Screen

6. Charger unit

7. Power Cable

8. Microwave Connection Point

9. Open-ended Coaxial/Contact Probe

10. Probe Cable Holding arm/handle

11. Inner Conductive Live End

12. Dielectric Material

13. Outer Conductor/Ground Connection

14. Inner Conductive Live End-Port 1

15. Dielectric Material Type 1

16. Dielectric Material Type 2

17. Inner Conductive Live End-Port 2

18. Dielectric Material-Type 3

19. Biopsy Guide

20. Resonator

21. Planar Microstrip Resonator

22. Planar ring resonator

23. Narrow Band Antenna

24. Patch

25. Substrate

26. SMA connector

### DETAILED DESCRIPTION OF THE EMBODIMENTS

The tissue identification device (1) subject to the invention comprises;
RF/microwave cable (3) located on the tissue identification device (1),
a microwave connection point (8) that is located near the tissue identification device (1), and that enables connection to the device with the RF/microwave cable (3),
a measurement device (2) that is located near the tissue identification device (1), and that is connected to the device with the RF/microwave cable (3),
an S parameter measurement unit and calculation/retrieval (computer) unit (4) located on the tissue identification device (1),
a digital screen (5) disposed in the S parameter measurement unit and calculation unit (4), which is located at the front section of the tissue identification device (1),
a charger unit (6) on which the tissue identification device (1) and all other components are disposed on,
a power cable (1) that is connected from the back of the tissue identification device (7),
an open-ended coaxial/contact probe (9), located in the measurement tool (2) and at the end of the measurement tool (2),
a probe cable holding arm/handle (10) disposed in the measurement tool (2) that located between the open ended coaxial/contact probe (9) and the RF/Microwave Cable (3),
an inner conductive live end (11) that is located in the inner section of the open ended coaxial/contact probe (9),
a dielectric material (12) that surrounds the inner conductive live end (11) from the outside and that is located in the inner section of the open-ended coaxial/contact probe (9),
an outer conductor/ground connection (13) that surrounds the dielectric material (12) and the inner conductive live end (11) from the outside, and that is located in the inner section of the open-ended coaxial/contact probe (9),
an inner conductive live end-port 1 (14) that is located in the inner section of the open-ended coaxial/contact probe (9), during the dual port contact probe utilization as an open-ended coaxial probe for the tissue identification device (1),
a dielectric material type 1 (15) that surrounds the inner conductive live end-Port 1 (14) from the outside,
an outer conductor/ground connection (13) that surrounds the dielectric material type 1 (15) from the outside,
dielectric material type 2 (16) that surrounds the outer conductor/ground connection (13) from the outside,
an inner conductive live end-Port 2 (17) that surrounds the dielectric material type 2 (16) from the outside,
a dielectric material type 3 (18) that surrounds the inner conductive live end port 2 (17) from the outside,
an outer conductor/ground connection (13) that surrounds the dielectric material type 3 (18) from the outside.

Additionally, the invention can be expressed as a tissue analysis method wherein said method comprises the steps of;
A. starting the tissue identification device (1) via an on/off button
B. calibration of the tissue identification device (1) via a calibration unit
C. placement of the measurement tool (2) into the tissue under test.
D. measurement of the S parameters by the main measurement unit
E. decision given by the main measurement unit, for either carrying out a dielectric calculation from measured S parameter response or solely utilizing measured S parameters, according to the utilized measurement tool (2)
F. calculation of the dielectric constant/properties in the calculation unit/computer (4), using an inverse problem approach and data obtained from the S parameter measurement unit,
G. decision given by the S parameter measurement unit and the S parameter measurement (4) for either calculating input impedance from measured S parameters response or solely utilizing measured S parameters,
H. measurement of the input impedance by the S parameter measurement unit and the calculation unit/computer (4) located in the main measurement unit,
I. delivery of the collected/calculated data to the classification unit within the calculation unit/computer,
J. classification of the tissue under test, via the main measurement unit,
K. shutting off the tissue identification device (1) via an on/off button.

Operation principle of the tissue identification/characterization device (1) subject to the invention depends on the inherent dielectric property discrepancy observed between malignant and benign tissues at microwave frequencies. The tissue identification device (1) subject to the invention comprises an S parameter measurement unit, an open-ended coaxial/contact probe (9), a calculation unit (computer) (4) aimed to be utilized for processing the collected data, and a power supply. The differences between the tissues can be determined through retrieval of dielectric properties. These properties are usually calculated via measured S parameters. The retrieval can be performed according to the probe model and the measured S parameters. Alternatively, the tissue identification can be performed directly from the scattering parameters (S parameters). The dielectric properties of the material under test are not measurable quantities. The S₁₁ parameter response of an open-ended coaxial probe changes based on the dielectric properties of the material under test. In order for the dielectric properties to be calculated, the reflection coefficient (F) at the aperture of the probe is determined via the S parameters. To determine the reflection coefficient, the microwave connections, VNA and probe, can be modelled as an S parameter circuit with two gates. Based on this model the reflection coefficient can be expressed as follows;

$$Γ = \frac{{ρ}_{-} {s}_{11}}{ρ {s}_{22} + {s}_{12} {s}_{21} + {s}_{11} {s}_{22}} \qquad ( Equation I )$$

The S parameters shown in the above Equation I, are measured during calibration. The standard calibration is carried out with terminating the aperture of the probe with open, short and load. Alternatively, the calibration can be performed by collecting the S parameter measurement responses from three known liquids. The measured S parameters (ρ1, ρ2, ρ3) during calibration corresponds to reflection coefficients (Γ₁, Γ₂, Γ₃). Highly accurate dielectric property calculation is possible by maintaining the conditions that are present during calibration after it is completed. For example, loose microwave connections following calibration may affect the results or movement of the cable can potentially deteriorate the calibration. In fact, the probe and cable is fixed and the sample under test is raised to the tip of the probe in a laboratory environment to keep the calibration conditions. After the calibration step is completed, the impedance value measured when the probe is terminated with the material under test. The value is then normalized with the characteristic admittance of the line and it is correlated with the reflection coefficient as shown in Equation 1. Next, the admittance values are calculated from the measured reflection coefficients. Then the Debye and Cole-Cole parameters are retrieved. Note that these parameters represent dielectric properties namely permittivity and conductivity of the material under test for ultra-wideband frequencies. The retrieval of the parameters is performed via solution of Fredholm integral equations of the first degree. It should be noted that the Fredholm integral equations are ill-posed problem. Highly accurate calculation of dielectric properties can be obtained approaching this problem as an inverse problem and solving it with alternative methods such as iterative methods (Newton-Raphson) with Tikhonov regularization.

The dielectric property measurement procedure includes, first, connecting the commercial open-ended coaxial/contact probe (9) to an RF cable. Next, the cable is connected to a measurement unit; that is, to S parameter measurement unit which is similar to a vector or performance network analyser (VNA). Then the system is calibrated with the open-short-load calibration sequence. It should be noted that the probe is generally fixed to a position in a laboratory environment. After the calibration is completed, the material under test is placed to the aperture, namely the open end of the probe, to collect the measurement. The system calculates admittance and the dielectric properties (permittivity and conductivity) of the material based on the measured admittance. Several measurements must be performed on samples in order to separate the malignant and benign tissues and/or malignant and healthy/normal tissues from one another with high accuracy using the open-ended coaxial probe technique in a laboratory environment. The measurements taken with commercial probes in laboratory environment have an accuracy of 5%. Although the technique provides 95% accuracy in theory in a laboratory environment where a large number of measurements is usually performed on the same sample in order to verify the dielectric characteristics of the material, the applications envisaged within the scope of the invention cannot be realized due to high error rates that may arise in practical use. For an open-ended coaxial/contact probe (9) the reflection coefficient at the probe aperture is measured and the dielectric properties are calculated from this measurement through an admittance correlation. The calculation of probe admittance is performed measuring the probe reflection coefficient at the probe aperture or at the connector (VNA output) (26). The probe reflection coefficient is measured by the S parameter measurement unit and calculation unit (computer) (4) and then, the probe admittance is calculated from the measured S parameters during calibration. Principle assumption of the technique is that the aperture of the probe should be completely terminated by the sample under test. Establishment of a good contact for gel-like materials in laboratory environment, is enabled by applying a slight pressure on the material under test.

The tissue identification device (1) subject to the invention does not aim to perform microwave imaging and to process time domain signals. Moreover, the goal of the proposed techniques not a microwave image; therefore, it does not comprise a unit/software/algorithm system calculation unit to reach such aims. This technique finds the dielectric properties of the sample under test by solving the integral equations that define the correlation between measurable quantities and dielectric properties. The proposed solution includes numerical approximations obtained by measuring the S parameters instead of image outputs. The open ended coaxial/contact probe (9) is used to automatically and simultaneously decide if the tissue at the aperture of the probe is malignant or benign/healthy, by measuring the dielectric properties of prostate tissue during a biopsy, as malignant tissue exhibit different dielectric properties in comparison to benign/healthy tissue at microwave frequencies. It is expected that the measured S parameters changes depending on the dielectric properties of the tissues under test. It is known that the changes in the molecular structure of the tissues cause a dielectric property change. To this end, the dielectric properties of tissues with anomalies should be different from the dielectric properties of healthy tissues. Therefore, the reflected/absorbed power also varies and this enables the identification/characterization of tissues.

A problem that can potentially emerge during surgery is the accumulation of blood at the measurement area. Since blood has high dielectric properties, it may cause increase in the dielectric property measurement particularly of healthy or benign tissues. As a result, benign tissue can be mistakenly categorized as malignant tissue due to measured dielectric property change.

By using classification algorithms, (1) problems such as the accumulation of blood and/or body fluids in the measurement area, (2) user related errors of the measurement system, and (3) the insufficient termination of the probe aperture with tissue, can potentially be eliminated. A solution has been sought in prior devices for part of the above listed problems though the addition of mechanical properties to the method/device. However, proposed method in this work targets to eliminate above errors by collecting training data from clinical studies and feed the collected data to the classification algorithms. The supervised classification algorithms learn to distinguish the malignant and benign and/or malign and healthy/normal tissues with high accuracy. Therefore, a system can operate with an accuracy rate above 95%. This approach can enable classification of prostate tissue by a single and highly accurate measurement.

In an alternative embodiment of the invention, a source/measurement unit transmits the microwave signals via a probe to the material under test and receives the scattered signal from the material via the probe. The dielectric properties of material are calculated using an inverse problem approach algorithm and the calculated dielectric properties are given to a machine learning algorithm with automated decision making capabilities. Hermetically sealed measurement unit can be operated with a battery and can be completely sterilized for surgical operations.

Wide band antennas, narrow band antennas (23) resonators (20) and open-ended coaxial/contact probes (9) can potentially be utilized as a probe and connected to S parameter measurement unit and calculation unit (4). When wide band antennas, narrow band antennas (23), and resonators (20) are used as probes, the tissue characterization can be performed either from the calculated dielectric properties, extracted by certain numerical approaches at determined frequencies, or directly from the measured reflection coefficient. Next, the collected reflection coefficient or dielectric properties can be given to machine learning algorithms for diagnosis of prostate cancer in a layered tissue medium.

In an alternative embodiment of the invention, the measurement unit, is connected to the open ended coaxial/contact probe (9) that can receive or submit data via microwaves. The probe is fabricated with a permanent connection to the RF cable; that is, without an SMA connection. This probe may have a single connection point where S₁₁ parameters are measured or for two port connection point where both S₁₁ and S₂₂ parameters are measured. The probe and cable can be sterilized.

In another alternative embodiment of the invention the measurement unit where S₁₁ or S₂₁ or both parameters can be measured together. S parameter measurement unit is connected to a planar antenna or a resonator, and these measurements are fed directly to the machine learning algorithm.

In another alternative embodiment of the invention, the probe or resonator (20) is directly connected to the S parameter measurement unit and calculation unit/computer (4). That is, without a cable. Consequently, the probe is not affected by the cable movement during measurements.

What is claimed is:

1. A device developed for a diagnosis of malignant prostate tumour tissues to be used in a healthcare sector, comprising:
an RF/microwave cable located on a tissue identification device,
a charger unit, wherein the tissue identification device and all other/related components are disposed on the charger unit,
a power cable connected to a back of the tissue identification device,
an open-ended coaxial/contact probe, located at a tip of a measurement tool,
an inner conductor (live end) located at a center of the open-ended coaxial/contact probe,
a dielectric material surrounding the inner conductor (live end) sandwiched between the inner conductor and an outer conductor of the open-ended coaxial/contact probe,
an outer conductor/ground connection surrounding the dielectric material and the inner conductor (live end) located in an inner section of the open-ended coaxial/contact probe,
a first inner conductor (live end) port located at the center of the open-ended coaxial/contact probe, to be employed during a dual port application of the tissue identification device,
a first dielectric material type surrounding the first inner conductor (live end) port,
the outer conductor/ground connection surrounding the first dielectric material type,
a second dielectric material type surrounding the outer conductor/ground connection,
a second inner conductor (live end) port surrounding the second dielectric material type,
a third dielectric material type surrounding the second inner conductor (live end) port,
the outer conductor/ground connection surrounding the third dielectric material type,
a measurement device located at a left side of the tissue identification device and connected to the device with the RF/microwave cable,
an S parameter measurement unit and calculation unit located on the tissue identification device.

2. The device according to claim 1, further comprising a microwave connection point located near the tissue identification device and enabling a connection to the device with the RF/microwave cable.

3. The device according to claim 1, further comprising a digital screen disposed in the S parameter measurement unit and calculation unit located at a front section of the tissue identification device,

4. The device according to claim 1, further comprising a probe cable holding arm/handle located within the measurement tool and located between the open-ended coaxial/contact probe and the RF/microwave cable.

5. A tissue analysis method comprising:
starting an tissue identification device via an on/off button,
calibration of the tissue identification device by a calibration unit,
placement of a measurement tool (2) into a tissue to be identified, and measurement of S parameters by a main measurement unit,
deciding by the main measurement unit, for carrying out a dielectric calculation or using the S parameters instead, according to a type of the measurement tool,
calculation of a dielectric constant in a calculation unit (computer), using an inverse problem approach and data obtained from a S parameter measurement unit,
deciding through the S parameter measurement unit and the calculation unit (computer) whether to calculate an input impedance or use the S parameters instead,
measurement of the input impedance via the S parameter measurement unit and the calculation unit (computer) located in the main measurement unit,
delivery of the data collected to a classification unit within the calculation unit (computer),
classification of the tissue under a test via the main measurement unit,
shutting off the tissue identification device via the on/off button.
